(19) United States
(12) Patent Application Publication
(10) Pub. No.: US 2017/0025709 A1
(43) Pub. Date: Jan. 26, 2017

(54) IMPROVING THE ION CONDUCTIVITY OF AN ELECTROLYTE BASED ON LITHIUM IMIDAZOLATE SALTS

(71) Applicant: ARKEMA FRANCE, Colombes (FR)

(72) Inventor: Gregory SCHMIDT, Saint Andeol Le Chateau (FR)

(73) Assignee: ARKEMA FRANCE, Colombes (FR)

(21) Appl. No.: 15/125,793

(22) PCT Filed: Mar. 9, 2015
(86) PCT No.: PCT/FR2015/050574

§ 371 Date: Sep. 13, 2016

(30) Foreign Application Priority Data
Mar. 14, 2014 (FR) 1452145
May 30, 2014 (FR) 1454902

(51) Int. Cl. H01M 10/0569; C07D 233/90; H01M 10/0525

(52) U.S. Cl. CPC H01M 10/0569; H01M 10/0525; C07D 233/90; H01M 2300/0037

(57) ABSTRACT

The invention relates to a composition comprising at least one electrolyte based on lithium imidazolate salts and to the use of nitrile or dinitrile solvents to increase the ion conductivity of electrolyte based on lithium imidazolate salts. The invention also relates to the use of the electrolyte composition in Li-ion batteries.

### FIELD OF THE INVENTION

The present invention relates to a composition comprising at least one electrolyte based on lithium imidazolate salts and to the use of nitrile or dinitrile solvents for increasing the ionic conductivity of an electrolyte based on lithium imidazolate salts. Another subject matter of the present invention is the use of the electrolytic composition in Li-ion batteries.

### TECHNICAL BACKGROUND

A lithium-ion battery comprises at least one negative electrode (anode), one positive electrode (cathode), one separator and one electrolyte. The electrolyte generally consists of a lithium salt dissolved in a solvent which is generally a mixture of organic carbonates, in order to have a good compromise between the viscosity and the dielectric constant. Additives can subsequently be added in order to improve the stability of the electrolyte salts.

The most widely used salts include lithium hexafluorophosphate (LiPF₆), which has many of the numerous qualities required but exhibits the disadvantage of decomposing in the form of hydrofluoric acid gas by reaction with water. This presents safety problems, in particular in the context of the nearby use of lithium-ion batteries for specific vehicles.

Recently, other salts have been developed, such as LiTDI (lithium 1-trifluoromethyl-4,5-dicyanoimidazolate) and Li PDI (lithium 1-pentafluoroethyl-4,5-dicyanoimidazolate). These salts exhibit the advantages of having fewer fluorine atoms and of having strong carbon-fluorine bonds instead of the weaker phosphorus-fluorine bonds of LiPF₆.

Furthermore, the patent WO2010023413 shows that these salts exhibit conductivities of the order of 6 mS/cm, a very good dissociation between the imidazolate anion and the lithium cation and their use as electrolyte salt of Li-ion batteries. However, this ionic conductivity, measured in “conventional” solvents for electrolytes, which are the mixtures of carbonates, is too low for use in batteries referred to as of power type.

The applicant has discovered that the use of a solvent exhibiting at least one nitrile functional group makes it possible to improve the ionic conductivity of these lithium salts.

### DESCRIPTION

The invention relates first to an electrolytic composition comprising one or more lithium salts of formula (A) in a solvent comprising at least one nitrile functional group or a mixture of solvents, at least one of which comprises a nitrile functional group.

Another subject matter of the invention is the use of said electrolyte as electrolyte for Li-ion batteries.

An additional subject matter of the invention is the use of a solvent comprising at least one nitrile functional group in order to improve the ionic conductivity of an electrolyte based on lithium imidazolate salts.

The lithium salts of formula (A) are represented below:

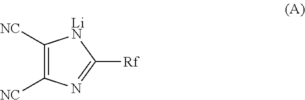

with Rf representing a fluorine atom, a nitrile group, an optionally fluorinated or perfluorinated alkyl group having from 1 to 5 carbons, an optionally fluorinated or perfluorinated alkoxy group having from 1 to 5 carbons or an optionally fluorinated or perfluorinated oxa-alkoxy having from 1 to 5 carbons.

Mention may in particular be made, by way of examples, of the following Rf groups: F, CF₃, CHF₂, CH₂F, C₂HF₄, C₂H₂F₃, C₂H₃F₂, C₂F₅, C₃F₇, C₃H₂F₅, C₃H₄F₃, C₄F₉, C₄H₂F₇, C₄H₄F₅, C₅F₁₁, C₃F₅OCF₃, C₂F₄OCF₃, C₂H₂F₂OCF₃, CF₂OCF₃, C₅F₁₁OCH₃, CF₂OC₂H₅, CF₂OC₂H₄OCH₃, CF₂OC₂H₄OC₂H₅, CF₂OCH₂OCF₃, CF(CF₃)OCH₃, CF(CF₃)OC₂H₅, CF(CF₃)OC₂H₄OCH₃ or CF(CF₃)OC₂H₂F₃.

Preferably, Rf represents CF₃.

The present invention makes it possible to overcome the disadvantages of the salts described above. This is because, although these salts are particularly advantageous as a result of their chemical and electrochemical stability and of the high separation between the anion and the lithium cation, their low ionic conductivity, however, limits their performance in terms of power. This low conductivity can be attributed not only to a low dissociation of the salt in conventional electrolyte solvents, in particular carbonates, but also to a high viscosity due to the size of the anion. Mention may in particular be made, as carbonates conventionally used, of ethylene carbonate, dimethyl carbonate, ethyl methyl carbonate, diethyl carbonate or propylene carbonate.

The applicant has discovered that the use of a solvent comprising at least one nitrile functional group in the presence or absence of cosolvent(s) makes it possible to greatly improve the ionic conductivity of the electrolytes prepared from the lithium salts of formula (A). Without being committed to any one explanation, the applicant believes that the nitrile functional group, because of its flatness and its affinity for the nitrile functional groups of the anion of the salt of formula (A), makes possible better dissociation of the lithium salt. Furthermore, the solvents comprising at least one nitrile functional group have advantages compared with the conventional solvents. Thus, the low viscosity and the wide temperature range in which the solvents comprising at least one nitrile functional group are in the liquid state provide a broader operating temperature range of the battery.

The solvents comprising at least one nitrile functional group according to the present invention can be represented by the general formula R(CN)ₓ where x is a number between 1 and 3 and R represents an optionally fluorinated or perfluorinated alkyl group having from 1 to 5 carbons, an optionally fluorinated or perfluorinated alkoxy group having from 1 to 5 carbons or an optionally fluorinated or perfluorinated oxa-alkoxy having from 1 to 5 carbons, the solvents preferably being aprotic, such as propionitrile.

According to one embodiment, x is equal to 2 and R has the same meaning as above. Glutaronitrile, methoxyglutaronitrile, 2-methylglutaronitrile, 3-methylglutaronitrile, adiponitrile and malononitrile are preferred.

Mention may in particular be made, as solvents comprising at least one nitrile functional group, of acetonitrile, pyruvonitrile, propionitrile, methoxypropionitrile, dimethylaminopropionitrile, butyronitrile, isobutyronitrile, valeronitrile, pivalonitrile, isovaleronitrile, glutaronitrile, methoxyglutaronitrile, 2-methylglutaronitrile, 3-methylglutaronitrile, adiponitrile and malononitrile.

The nitrile solvent can be used alone or as a mixture with one or five cosolvents.

Mention may in particular be made, as cosolvents, of the above-mentioned nitriles of formula R(CN)ₓ, carbonates, such as ethylene carbonate, dimethyl carbonate, ethyl methyl carbonate, diethyl carbonate or propylene carbonate, or glymes, such as ethylene glycol dimethyl ether, diethylene glycol dimethyl ether, dipropylene glycol dimethyl ether, diethylene glycol diethyl ether, triethylene glycol dimethyl ether, diethylene glycol dibutyl ether, tetraethylene glycol dimethyl ether and diethylene glycol t-butyl methyl ether.

Preferably, the solvent(s) comprising at least one nitrile functional group represent(s) between 1% and 100% by volume of all of the solvents in the electrolytic composition, advantageously between 10 and 90% by volume.

More particularly, in the presence of solvents of formula R(CN)ₓ in which x is equal to 2 or 3, the cosolvent or cosolvents is or are, preferably, chosen from dimethyl carbonate, ethyl methyl carbonate, diethyl carbonate, propylene carbonate, ethylene glycol dimethyl ether, diethylene glycol dimethyl ether, dipropylene glycol dimethyl ether, diethylene glycol diethyl ether and triethylene glycol dimethyl ether.

According to this embodiment (that is to say, when x=2 or 3), the proportion by volume of solvent(s) comprising at least one nitrile functional group R(CN)ₓ in the mixture of solvents is preferably between 1% and 50% and the proportion by volume of the sum of the cosolvents is preferably between 50% and 99% of the total volume of the mixture.

According to another embodiment, in the presence of solvents of formula R(CN)ₓ in which x is equal to 1, the cosolvent or cosolvents is or are, preferably, chosen from ethylene carbonate, propylene carbonate, diethylene glycol dibutyl ether, tetraethylene glycol dimethyl ether and diethylene glycol t-butyl methyl ether.

According to this embodiment (that is to say, when x=1), the proportion by volume of solvent(s) comprising at least one nitrile functional group R(CN)ₓ in the mixture of solvents is preferably between 50% and 99% and the proportion by volume of the sum of the cosolvents is preferably between 1% and 50% of the total volume of the mixture.

The amount of lithium salt of formula (A) dissolved in the mixture of solvents described above can vary between 0.01 and 10 mol/l, more preferably between 0.05 and 2 mol/l.

The amount of lithium salt of formula (A) present in the electrolytic composition according to the present invention can vary between 0.01 and 10 mol/l, preferably between 0.05 and 2 mol/l.

Preferably, the lithium salt(s) of formula (A) represent(s) between 2% and 100% by weight of all of the salts present in the electrolytic composition, advantageously between 25% and 100% by weight.

Another subject matter of the present invention is the use of at least one solvent comprising at least one nitrile functional group in order to improve the ionic conductivity of an electrolyte based on lithium imidazolate salts preferably of formula (A).

The amount of solvent comprising at least one nitrile functional group involved is preferably that indicated above.

The nitrile solvent is preferably chosen from the list described above.

An additional subject matter of the present invention is the use of the abovementioned compositions as electrolyte for Li-ion batteries.

### EXAMPLES

The following examples illustrate the invention without limiting it. In the following examples, the ionic conductivities were measured by impedance spectroscopy using a conductivity cell provided with two plates made of platinized platinum.

### Example 1

An electrolyte containing 1 mol/l of a salt of formula (A) where Rf═CF₃ (LiTDI) in propionitrile is prepared. The ionic conductivity of this electrolyte is measured using the technique described above. The value obtained is 12 mS/cm.

### Example 2

An electrolyte containing 0.9 mol/L of a salt of formula (A) where Rf═CF₃ (LiTDI) in an ethylene carbonate/propionitrile mixture with different proportions by volume is prepared. The ionic conductivities of these electrolytes are measured using the technique described above. The values obtained are summarized in the following table.

| EC/propionitrile ratio | Ionic |
|---|---|
| by volume | conductivity |
| 10/90 | 10.44 |
| 20/80 | 9.91 |
| 30/70 | 9.25 |
| 40/60 | 8.54 |
| 50/50 | 7.76 |

1. An electrolytic composition comprising at least one lithium salt of formula (A): 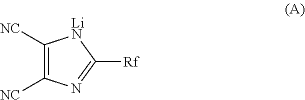
in which Rf represents a fluorine atom, a nitrile group, an optionally fluorinated or perfluorinated alkyl group having from 1 to 5 carbons, an optionally fluorinated or perfluorinated alkoxy group having from 1 to 5 carbons or an optionally fluorinated or perfluorinated oxa-alkoxy having from 1 to 5 carbons,
in a solvent comprising at least one nitrile functional group or a mixture of solvents, at least one of which comprises a nitrile functional group.

2. The composition as claimed in claim 1, characterized in that the solvent comprising at least one nitrile functional group is represented by the general formula R(CN)ₓ where x is a number between 1 and 3 and R represents an optionally fluorinated or perfluorinated alkyl group having from 1 to 5 carbons, an optionally fluorinated or perfluorinated alkoxy group having from 1 to 5 carbons or an optionally fluorinated or perfluorinated oxa-alkoxy having from 1 to 5 carbons, preferably a solvent which is aprotic.

3. The composition as claimed in claim 1, characterized in that Rf represents F, CF₃, CHF₂, CH₂F, C₂HF₄, C₂H₂F₃, C₂H₃F₂, C₂F₅, C₃F₇, C₃H₂F₅, C₃H₄F₃, C₄F₉, C₄H₂F₇, C₄H₄F₅, C₅F₁₁, C₃F₅OCF₃, C₂F₄OCF₃, C₂H₂F₂OCF₃, CF₂OCF₃, C₅F₁₁OCH₃, CF₂OC₂H₅, CF₂OC₂H₄OCH₃, CF₂OC₂H₄OC₂H₅, CF₂OCH₂OCF₃, CF(CF₃)OCH₃, CF(CF₃)OC₂H₅, CF(CF₃)OC₂H₄OCH₃ or CF(CF₃)OC₂H₂F₃.

4. The composition as claimed in claim 3, characterized in that Rf represents CF₃.

5. The composition as claimed in claim 1, characterized in that the amount of lithium salt of formula (A) present in the electrolytic composition according to the present invention can vary between 0.01 and 10 mol/l, preferably between 0.05 and 2 mol/l.

6. The composition as claimed in claim 1, characterized in that the solvent(s) comprising at least one nitrile functional group represent(s) between 1% and 100% by volume of all of the solvents in the electrolytic composition.

7. The composition as claimed in claim 2, characterized in that x is equal to 2.

8. A Li-ion battery comprising a composition as claimed in claim 1.

9. A method of improving ionic conductivity of an electrolyte based on lithium imidazolate salts method comprising using in the electrolyte a solvent comprising at least one nitrile functional group or a mixture of solvents, at least one of which comprises a nitrile functional group of formula (A).

10. The method as claimed in claim 9, characterized in that the nitrile solvent is represented by the general formula R(CN)ₓ where x is a number between 1 and 3 and R represents an optionally fluorinated or perfluorinated alkyl group having from 1 to 5 carbons, an optionally fluorinated or perfluorinated alkoxy group having from 1 to 5 carbons or an optionally fluorinated or perfluorinated oxa-alkoxy having from 1 to 5 carbons, preferably aprotic.

11. The method as claimed in claim 9, characterized in that x is equal to 2.

12. The method as claimed in claim 9 in a Li-ion battery.
